(19) United States
(12) Patent Application Publication
(10) Pub. No.: US 2005/0232809 A1
(43) Pub. Date: Oct. 20, 2005

(54) Radiator module with sterilizing device

(75) Inventor: Hsin Mao Hsieh, Ping Tung City (TW)

Correspondence Address: BACON & THOMAS, PLLC, 625 SLATERS LANE, FOURTH FLOOR, ALEXANDRIA, VA, 22314, US

(73) Assignee: ADDA CORPORATION, Ping Tung City (TW)

(21) Appl. No.: 10/827,233

(22) Filed: Apr. 20, 2004

(51) Int. Cl. A61L 2/00

(52) U.S. Cl. 422/24; 422/121

(57) ABSTRACT

Air blown into and drawn out of an electronic product for dissipating heat generated by the product is sterilized by a sterilizing device mounted on an oblique planar surface of a pedestal secured to a side of the product.

### BACKGROUND OF THE INVENTION

I. Field of the Invention

The present invention relates generally to a radiator module and, more specifically, to a radiator module with sterilizing device that sterilizes bacteria while radiating heat.

II. Description of the Prior Art

Heretofore, it is known that more and more air-contagious diseases appear. In addition to the widely-known flu, these also include SARS that attacked the world last year and the bird flu that is attacking some regions this year, which has caused much concern about the ever-deteriorating air quality around us. While paying much attention to keeping living environment and surrounding environment clean and sterilized, people fail to pay attention to the adverse impact resulting from the use of electronic products and computers in particular.

As shown in FIG. 1, a conventional desktop computer usually comprises a case; at the rear of the case is a power supply device that comprises a fan; the case further comprises a radiator module used to radiate heat for CPU. However, the fans on power supply device and radiator module both draw cold air into the case through the air inlet at the front section of the case and sends out hot air from the rear of the case.

Generally, computers are used in closed central air-conditioned environments and their fans tend to collect dust. Therefore, virus carried by any staff member or other people into the environment may stay in the environment and get collected by fans. In addition, the back of the case is usually close to walls or some shelters. As a result, air sent out of the back of the case is usually bounced by walls or shelters and directly inhaled by computer operators, thus increasing the possibility of disease attack for computer operators.

### SUMMARY OF THE INVENTION

It is therefore a primary object of the invention to provide a radiator module with sterilizing device that comprises a pedestal, the pedestal has fans; a sterilizing device is installed on the pedestal; the sterilizing device can be optical media, ozone and other sterilizing devices can replace UV lamp. In practical use, the pedestal is installed on the proper location of the electronic product; the fans on the pedestal inhale external air into the electronic product, while the hot air is sending out the sterilizing device on the electronic product kills bacteria or virus carried by incoming and outgoing air The mechanism effectively eliminates bacteria, virus and keeps them from spreading around the electronic product which effectively prevents outgoing air from directly flowing towards computer operators, this prevents bacteria from propagation and contagion and thus ensures health for computer operators.

### BRIEF DESCRIPTION OF THE DRAWINGS

The accomplishment of the above-mentioned object of the present invention will become apparent from the following description and its accompanying drawings which disclose illustrative an embodiment of the present invention, and are as follows:

FIG. 1 is a perspective view of the prior art;

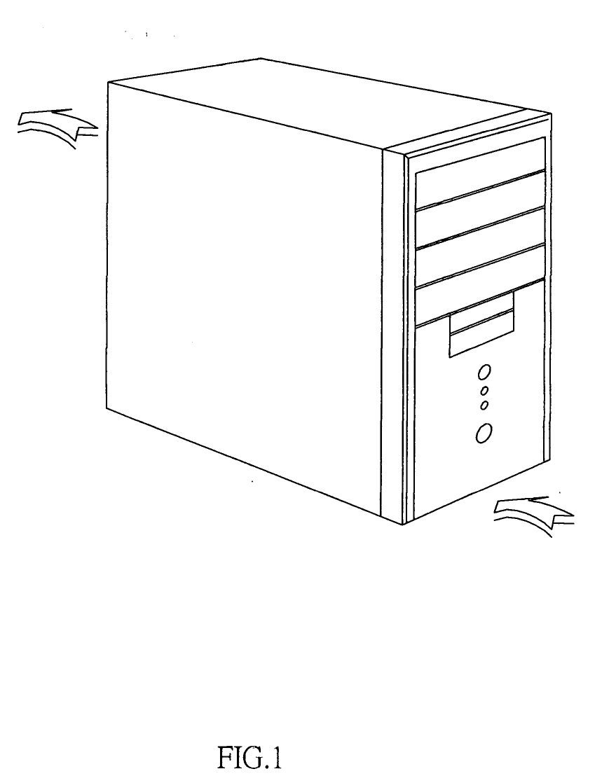

FIG. 2 is an assembly view of the present invention;

FIG. 3 is a perspective view of the present invention;

FIG. 4 is an application view of the present invention.

### DESCRIPTION OF THE PREFERRED EMBODIMENT

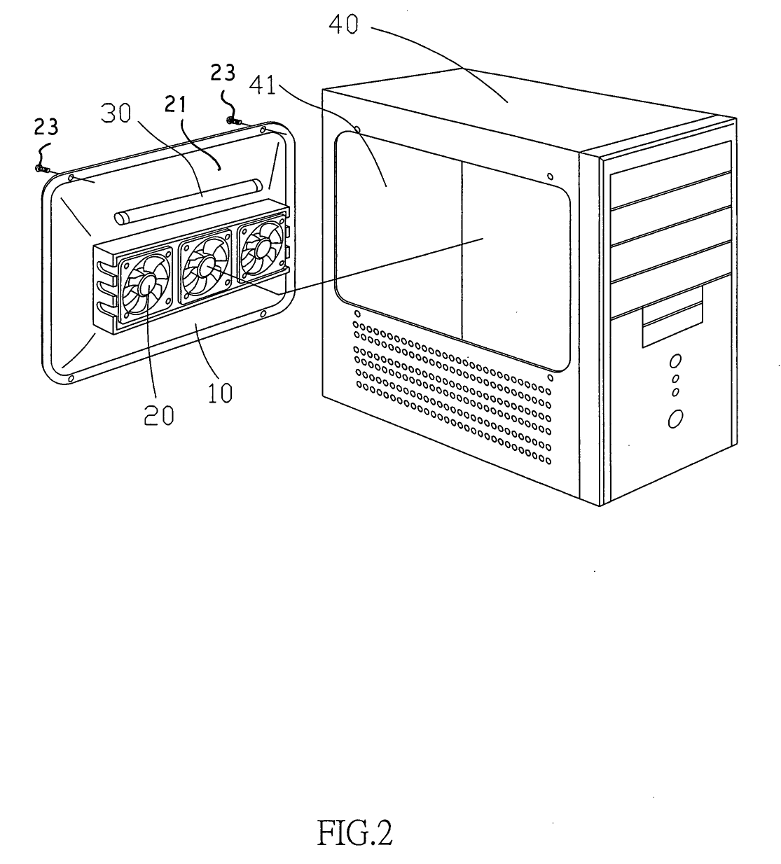

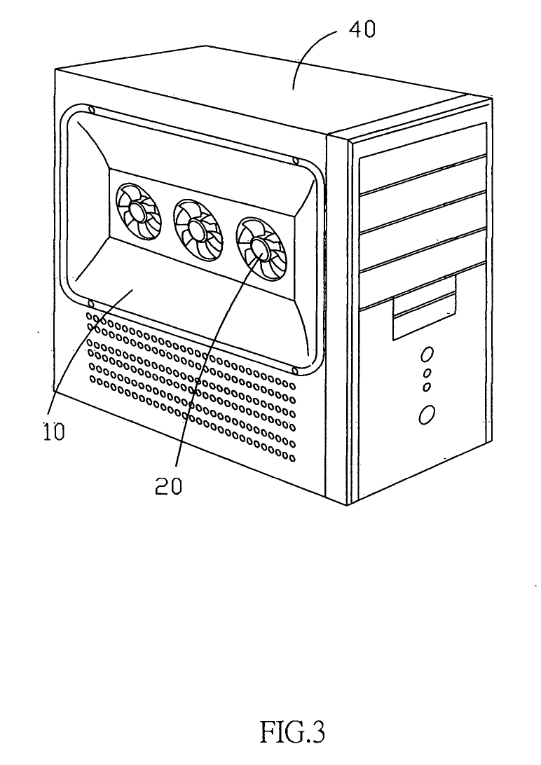

As shown in FIGS. 2 and 3, the present invention is installed on an electronic product 40. In the present embodiment, the electronic product 40 is a desktop computer that comprises an integrated pedestal 10; the pedestal 10 is in flat shape and comprises a minimum of two fans 20 (three fans in the present embodiment) that are installed on a radiator base (not shown in Fig.) made with radiating materials; the section on the pedestal 10 unoccupied by fans 20 forms a oblique plain on which a sterilizing device 30 is installed. The sterilizing device 30 in the present embodiment is a UV lamp. Optical media, ozone and other sterilizing devices can replace UV lamp.

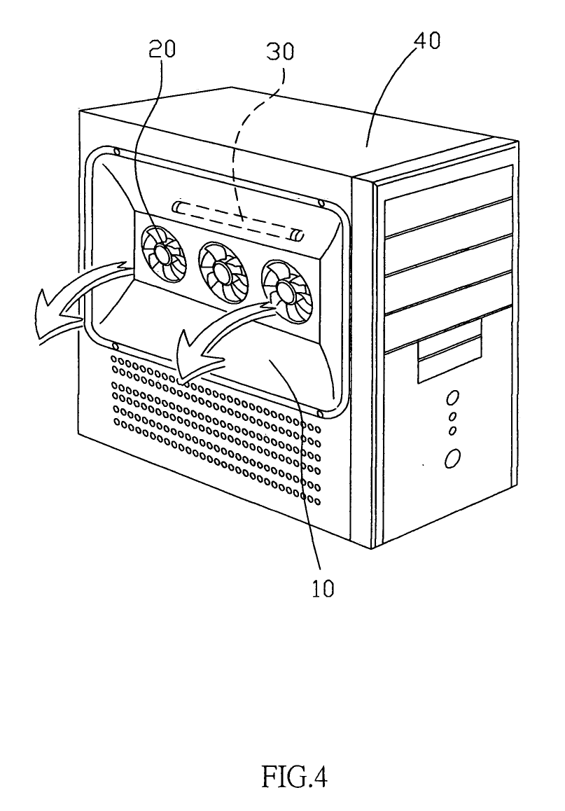

In practical use, as shown in FIGS. 2, 3 and 4, an opening 41 is on one side of the electronic product 40; fix the pedestal 10 in the opening 41 on the electronic product 40 and use a coupling device to fix the pedestal 10 on the side of the electronic product 40; switch on power to activate fans 20 on the pedestal 10 such that the sterilizing device 30 on the electronic product 40 kills bacteria or virus carried by incoming and outgoing air while fans 20 are drawing cold air into and sending hot air out of the electronic product 40. The mechanism effectively eliminates bacteria, virus and keeps them from spreading around the electronic product 40. In addition, the pedestal 10 is installed on the electronic product 40, which effectively prevents outgoing air from directly flowing towards computer operators. As a result, this prevents bacteria from propagation and contagion and thus ensures health for computer operators.

While a preferred embodiment of the invention has been shown and described in detail, it will be readily understood and appreciated that numerous omissions, changes and additions may be made without departing from the spirit and scope of the invention.

1-5. (canceled)

6. A radiator module with sterilizing device comprising:
(a) a pedestal for installation on a side of an electronic product, the pedestal having a first portion defining an oblique planar surface and a second portion;
(b) at least one fan mounted on the second portion of the pedestal for blowing air into and drawing air out of the electronic product to dissipate heat generated by the product; and
(c) a sterilizing device mounted on the oblique planar surface of the pedestal for sterilizing the air blown into and drawn from the electronic product by the at least one fan.

7. The radiator module of claim 6, wherein the pedestal includes two fans.

8. The radiator module of claim 6, wherein the electronic product is a computer.

9. The radiator module of claim 6, wherein the sterilizing device is an ultraviolet lamp.

10. The radiator module of claim 6, further including a coupling device for securing the pedestal on the side of the electronic product.
